(19) United States
(12) Patent Application Publication
(10) Pub. No.: US 2004/0210399 A1
(43) Pub. Date: Oct. 21, 2004

(54) Random number generator method and system for genetic algorithm analysis

(76) Inventors: J. Barry Shackleford, Portola Valley, CA (US); Motoo Tanaka, Tokyo (JP)

Correspondence Address: HEWLETT-PACKARD DEVELOPMENT COMPANY, Intellectual Property Administration, P.O. Box 272400, Fort Collins, CO, 80527-2400, US

(21) Appl. No.: 10/413,779

(22) Filed: Apr. 15, 2003

(51) Int. Cl. G06F 19/00; G01N 33/48; G01N 33/50

(52) U.S. Cl. 702/20

(57) ABSTRACT

A method and apparatus is used to initialize a population memory of electronic chromosomes for use in genetic algorithm (GA) analysis. Initializing the population memory includes providing an initial state into a first parent chromosome and a second parent chromosome, entering a current state of a first parent chromosome and a second parent chromosome into a cellular automata, selecting a neighbor state of the second parent chromosome for entry into the cellular automata, and generating a random number sequence using the cellular automata for use in the population memory.

### BACKGROUND OF THE INVENTION

The present invention relates to the use of genetic algorithms (GA) as a solution methodology to various computation intensive problems.

GA uses an electronic chromosome to represent a potential answer to a problem being solved. The electronic chromosome is typically a binary string of “0s” and “1s” that identifies each electronic chromosome used by the GA analysis. These chromosome values are stored in population memory where they can be accessed and evaluated during GA analysis.

It is important that the population memory is initialized with high-quality random numbers at the beginning of the GA analysis. By entering random numbers in the population memory, the GA analysis has a wider range of solutions to evaluate and is more likely to converge upon an optimal or near optimal solution. The GA analysis may access the population memory thousands of times as different pairs of parent chromosomes are evaluated. These parent chromosomes are recombined and altered through the crossover and mutation operations to produce child chromosomes. If a resulting child chromosome evaluated according to a fitness function has a higher fitness or lower cost than a parent chromosome, the child chromosome replaces the parent chromosome in the population memory.

Over time, the random numbers entered initially in the population memory are altered and replaced with more optimal or near optimal solutions to the problem being analyzed. Although the initial chromosomes entered into population memory often do not survive, they greatly influence the optimal or near optimal solutions produced. The higher the quality of randomness in the initial chromosomes, the greater the potential for covering a wider range of solutions and converging on the optimal solution. Conversely, if the numbers are not truly random, certain ranges of the solution space may not be explored through the GA analysis. Optimal or even near optimal solutions may not be found as the initial values were not high-quality random values and certain ranges of values were omitted.

Random number generation is also important when designing hardware and implementing the GA circuitry. In general, the GA analysis circuitry should be scalable, flexible, and cost-effective. Unfortunately, conventional random number generator circuits using multiplexers and other gates with extremely wide bus widths are very expensive to implement and layout on a chip. Because conventional random number generators are not integrated into the GA analysis circuitry, additional bus lines and circuitry must connected to the population memory. This increases costs while decreasing scalability of hardware as the problems being solved using GA analysis circuitry become more complex.

### BRIEF DESCRIPTION OF THE DRAWINGS

FIG. 1 is a flow chart diagram of the operations for performing genetic algorithm (GA) analysis in accordance with one implementation of the present invention;

FIG. 2 is a block diagram illustrating both the cross-over operation between parent chromosomes and the mutation operation on a child chromosome in accordance with one implementation of the present invention;

FIG. 3 is block diagram representation of a GA circuit designed in accordance with one implementation of the present invention;

FIG. 4 is a block diagram illustrating a bit-slice designed to include the cellular automata random number generator (CA RNG) in accordance with one implementation of the present invention;

FIG. 5 is a flowchart diagram of the operations associated with generating the randomly generated numbers using cellular automata in accordance with one implementation of the present invention;

FIG. 6 provides a graphical representation of the random numbers generated without using cellular automata; and

FIG. 7 graphically illustrates a series of random numbers generated using cellular automata to develop random number sequences in accordance with one implementation of the present invention.

Like reference numbers and designations in the various drawings indicate like elements.

### SUMMARY OF THE INVENTION

One aspect of the present invention features a method of initializing a population memory of electronic chromosomes used in genetic algorithm analysis. Initializing the population memory includes providing an initial state into a first parent chromosome and an initial state into a second parent chromosome, entering a current state of a first parent chromosome and a current state of a second parent chromosome into a cellular automata, selecting a neighbor state of the second parent chromosome for entry into the cellular automata, and generating a random number sequence using the cellular automata for use in the population memory.

Another aspect of the present invention describes an apparatus for initializing a population memory of electronic chromosomes used in genetic algorithm analysis. The initialization apparatus includes a first parent circuit for receiving a parent chromosome from a population memory, a second parent circuit for receiving a parent chromosome from a population memory, and a cellular automata circuit that receives a first current state from a first electronic chromosome, a second current state from a second electronic chromosome, and a neighboring state from the second electronic chromosome.

### DETAILED DESCRIPTION

Aspects of the present invention are advantageous in at least one or more of the following ways. Implementations of the present invention use cellular automata (CA) to improve the quality of the random numbers generated and stored in the population memory used in genetic algorithm analysis. High quality random numbers improve the results produced through genetic algorithm analysis. For example, higher quality random numbers generated using CA in accordance with the present invention do not exhibit patterns and other artifacts often found when using conventional random number generation techniques. The resulting electronic chromosomes entered into a population memory have fewer inherent patterns and facilitate exploring potentially more optimal solutions that might otherwise have been overlooked.

In addition to identifying potentially more optimal solutions, implementations of the present invention make genetic algorithm circuitry more cost effective and efficient. Random number generation logic implemented using cellular automata is implemented as part of the bit-slice circuitry along with other bit-wise computational components used for genetic algorithm analysis. Integrating random number generation circuitry as part of a bit-slice makes it easier to scale the genetic algorithm circuitry and random number generation for different applications. Bit-slice genetic algorithm circuitry designed in accordance with the present invention can be chained together to create n-bit wide random numbers for the population memory along with the corresponding logic to process the corresponding n-bit wide chromosomes. Moreover, implementing random number generation and cellular automata in this bit-slice format avoids having to add separate additional bus lines sometimes spanning hundreds of bits and taking up precious chip surface area.

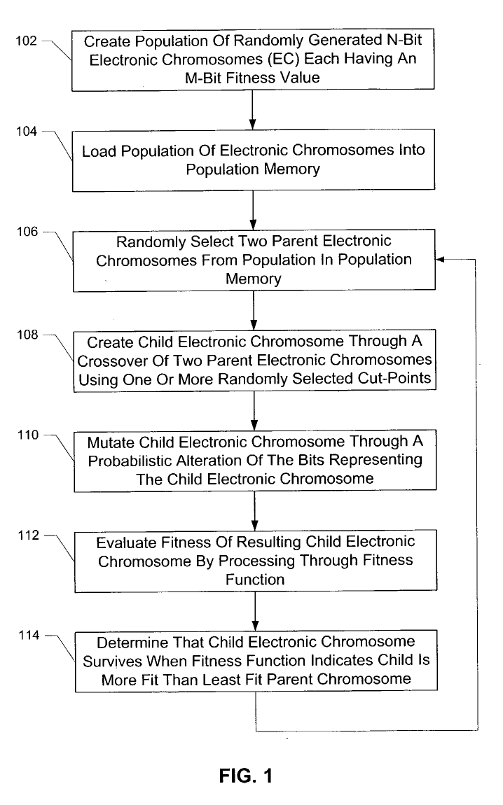

FIG. 1 is a flow chart diagram of the operations for performing genetic algorithm (GA) analysis in accordance with one implementation of the present invention. To begin GA analysis, a population of randomly generated n-bit electronic chromosomes (hereinafter referred to as chromosomes) is created and stored in population memory or other storage areas (102). Typically, the population memory also holds a fitness value corresponding to each of the n-bit chromosomes in the population. Each chromosome is evaluated by a fitness function and assigned a fitness value based on how well the chromosome appears to solve the problem being analyzed. Moreover, the fitness value determines which chromosomes will be kept in population memory and, eventually, the chromosome that most optimally solves the problem being analyzed.

The population memory is loaded with random n-bit binary patterns representing the chromosomes and corresponding m-bit fitness values assigned to each chromosome and related to the problem being studied (104). Implementations of the present invention initially fill the population memory with high-quality random numbers using cellular automata. Two of the chromosomes are selected at random from among the chromosomes in the population memory as a pair of parent chromosomes (one for each parent) (106). The corresponding fitness value from each new parent is compared with the fitness value of the current least-fit chromosome. If the comparison indicates the fitness value of the newly selected parent chromosome is less fit, than the selected parent chromosome becomes identified as the least fit parent or chromosome within the population memory. The pointer to the least fit parent or chromosome is maintained to facilitate rapid access and subsequent comparisons as needed.

A probabilistic crossover operation between the first and second parent chromosomes produces a child chromosome (108). One or more randomly selected cut points on the pair of chromosomes delineate the sections of the parent chromosome to be used in the creation of the child chromosome. Both parent chromosomes are cut at the same cut point(s) and combined together to create the new child chromosome. For example, a single cut point produces a child chromosome composed of left-cut portion of a first parent chromosome and the right-cut portion of a second parent chromosome.

While one implementation of the present invention uses a single cut-point, it is also possible that multiple cut-points are selected and used in creating the child chromosome. Further, it is also possible that no cut-point is selected in which case one parent chromosome is copied and used directly to create the new child chromosome. It should be appreciated that both location of the cut-point(s) and the decision to perform the cross-over occur probabilistically and are not predetermined.

The resultant child chromosome is mutated through a probabilistic alteration of the bits representing the child chromosome (110). In one implementation, a low-probability of I per-cent per bit is selected as the likelihood that a bit value will be mutated into another bit value. All bits have the same independent chance of mutation, so multiple bit changes in an n-bit chromosome are possible but less likely than a single-bit mutation. Typically, each bit in the child chromosome is mutated by inverting 0s to 1s and vice versa.

After the mutation operation, the child chromosome is evaluated and processed by a fitness function (112). Each fitness function is designed to solve different problems within the GA analysis framework and can be implemented in software, hardware, firmware, combinations thereof, and may include Very Large Scale Integration (VLSI) or Field Programmable Gate Array (FPGA) technologies, for example. To solve a new problem, a different fitness function can be designed and implemented within substantially the same GA analysis framework described herein. The fitness function processes the child chromosome and produces a fitness value indicating of how well the particular child chromosome solves the given problem.

The child chromosome and the corresponding fitness value are used to determine whether the child chromosome survives and potentially replaces a parent chromosome in the population memory (114). The fitness value associated with the child chromosome is compared with the fitness value corresponding to the least fit parent chromosome in the current population memory to determine if the child chromosome survives. If the survival comparison indicates the child chromosome is more fit than the least-fit parent chromosome, the child chromosome replaces the chromosome in the population memory corresponding to the least-fit parent chromosome. By repeating this process, the solution quality of the problem being solved by the GA increases as well as the overall fitness of the population.

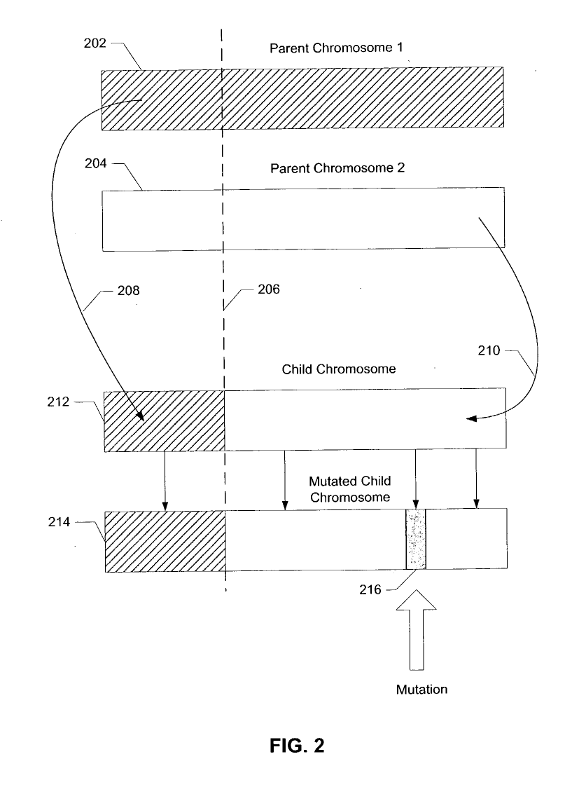

FIG. 2 is a block diagram illustrating both the cross-over operation between parent chromosomes and the mutation operation on a child chromosome in accordance with one implementation of the present invention. In this example, parent chromosome 202 and parent chromosome 204 are split along a single cut-point 206. Each parent contributes through cross-over operation 208 and cross- over operation 210 a portion of their electronic chromosome based on cut-point 206.

A child chromosome 212 having characteristics of both parent chromosomes is produced by these cross-over operations. Because the cut-point location is determined randomly, child chromosome 212 may have different proportions of each parent chromosome and is not limited to the combination illustrated herein. Multiple cut-points could also be used resulting in different portions of chromosomes from the parent chromosomes.

A mutation operation applied bit-wise to child chromosome 212 causes a probabilistic variation in binary representation of child chromosome 212. Although the probability of mutation is often low, the mutation helps explore other potential solutions or combinations that may not have existed or been available in the existing population memory. Mutation assists in rapid convergence on an optimal solution without testing every possible combination. In a protein sequencing problem for example, a mutation replaces a subfield of the child chromosome corresponding to one amino acid with another amino acid that may more closely solve the protein sequencing problem at hand.

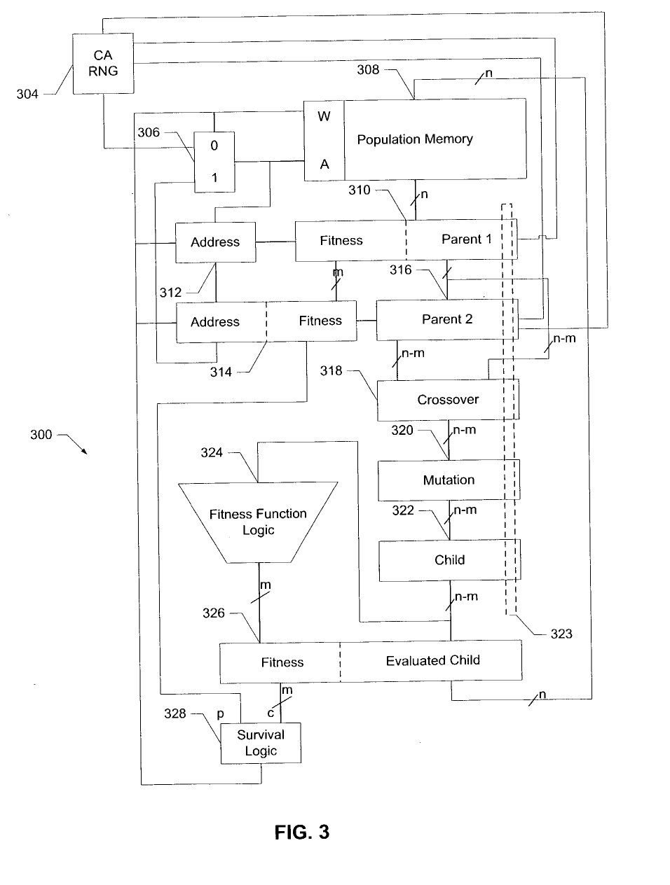

FIG. 3 is block diagram representation of a GA circuit 300 designed in accordance with one implementation of the present invention. GA circuit 300 includes a cellular automata random number generator (CA RNG) 304, a population memory MUX 306, a population memory 308, a parent 1 and fitness register 310, a parent 1 address register 312, a parent 2 address and fitness register 314, a parent 2 register 316, crossover logic 318, mutation logic 320, child register 322, a bit-slice CA/RNG 323 of the present invention, fitness function logic 324, evaluated child and fitness register 326, and survival logic 328.

During initialization mode, CA RNG 304 produces random numbers used for a variety of purposes in the GA circuitry including loading population memory 308 with initial population chromosomes. Additionally, CA RNG 304 can be used effectively to generate random addresses for selecting parents as well as random data to produce random crossover and mutation operations in the GA circuitry. In general, CA RNG 304 can be used for generating random numbers in the GA circuitry where scalability is not required and a bit-slice based CA/RNG of the present invention is not necessary or justified. MUX 306 facilitates storing the randomly generated addresses in population memory 308. In contrast, bit-slice CA/RNG 323 of the present invention generates random numbers for an n-bit wide chromosomes in population memory 308.

In running or operating mode, the genetic analysis circuit randomly selects from population memory 308 a chromosome, fitness value, and address for the first parent. These values are loaded into parent 1 and fitness register 310 and parent 1 address register 312. The genetic analysis circuit also randomly selects from population memory 308 a chromosome, fitness value, and address for the second parent chromosome and then loads them into parent 2 address and fitness register 314, and parent 2 register 316. To save clock cycles spent accessing memory, the contents of the register associated with parent 1 can also be shifted into parent 2 for subsequent iterations rather than loading parent 2 directly from memory.

Crossover logic 318 combines the parent 1 and parent 2 values in a probabilistic manner as previously described. Mutation of the resulting combination between parent 1 and parent 2 occurs, if at all, in mutation logic 320 and then is stored in child register 322 for further processing.

Each new child chromosome in child register 322 is also provided with a fitness value. Fitness function logic 324 processes the child chromosome stored in child register 322 according to the predetermined evaluation criteria to create the initial fitness value. This fitness value for the child chromosome is stored along with the child chromosome in evaluated child and fitness register 326 awaiting further processing/evaluation. In one implementation, the child fitness value in evaluated child and fitness register 326 is compared with the corresponding fitness of the lesser fit parent in population memory 308 based using survival logic 328. To locate the lesser fit parent more readily, the address of the lesser fit parent can be stored in a lesser-fit register. If the child chromosome has a better fitness than the lesser fit parent, it replaces the lesser fit parent and is stored at the lesser fit parent's address in population memory 308. Over time, the random numbers generated by an implementation of the present invention evolve into an optimal solution in accordance with the present invention and according to the GA analysis process.

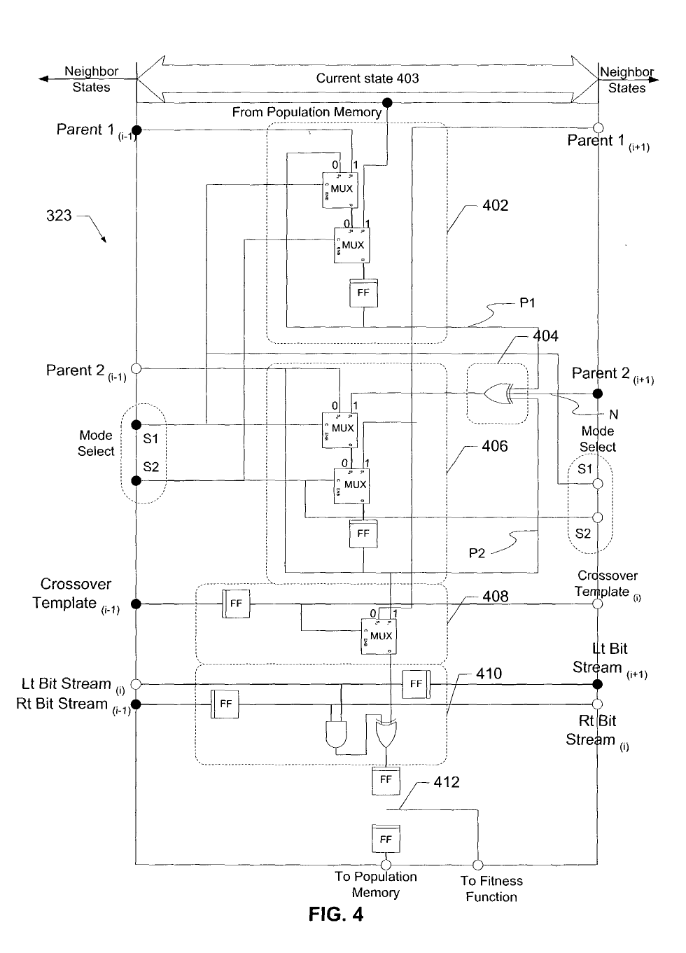

FIG. 4 is a block diagram illustrating a bit-slice designed to include the CA RNG in accordance with one implementation of the present invention. In this implementation, bit-slice CA/RNG 323 includes a Parent 1 402, a current state 403, a cellular automata (CA) 404, a parent 2 406, a crossover logic 408, a mutation logic 410, and child chromosome registers 412. Multiple bit-slices are connected together depending on the bit width of the electronic chromosome used in the GA circuit.

In the illustrated implementation of the present invention, bit-slice inputs are designated with an open circle (i.e., ◯) and bit-slice outputs are designated with a filled circle (i.e. ●). The number of bit-slices combined together depends, in part, on the width of the chromosome used in the GA circuit and the processing being performed on the various bits of information. For example, a 64 bit-wide chromosome could be implemented using 64 bit-slices designed in accordance with the present invention and illustrated in FIG. 4. Discrete logic elements used in the bit-slice implementation of the present invention include multiplexers (MUX), flip-flops (FF), exclusive-or (XOR), and AND logic elements and are identified accordingly in FIG. 4.

Prior to initializing the population memory, values are introduced initially to the first parent using a random right shifting serial number stream and the second parent using a random left shifting serial number stream. Parent 1 402 further includes circuitry that receives a bit at a particular offset from a chromosome identified as a first parent and retrieved from population memory. Similarly, parent 2 406 includes circuitry that receives a bit at a particular offset from another chromosome in population memory identified as a second parent. Current state 403 refers to the state information or data stored in flip-flops or other storage devices in different portions of bit-slice CA/RNG 323 of the present invention. Neighbor states refers to similar data or state information stored in adjacent circuitry that may be the same or similar to bit-slice CA/RNG 323 of the present invention.

In this implementation, cellular automata 404 is a circuit that receives and processes a first current state derived from the electronic chromosome in population memory identified as the first parent, a second current state derived from the electronic chromosome identified as the second parent, and a neighboring state from the second electronic chromosome at a bit-offset different from the first current state and second current state position described above. Cellular automata 404 provide a portion of the bit-slice that generates random numbers for the population memory in a scalable manner. Scalability allows the bit-slice and cellular automata used to generate random numbers to grow in correspondence with the chromosome width.

The first current state is at a particular offset from the first parent chromosome and the second current state is at a corresponding offset from the second parent chromosome. Neighboring state is at a particular offset in the second chromosome and may be different from the offsets associated with either the first or second current states. Cellular automata 404 operates on randomly generated right and left shifting bits as they pass through the first and second parent registers at the various offsets described above. Alternate implementations can use a variety of different random generators other than right or left shifting bit registers. Further, in alternate implementations, different offsets from either the first or second parent chromosomes are used depending on the suitability to the particular problem.

The example cellular automata illustrated in FIG. 4 is a three-input exclusive-or (XOR) function designed in accordance with the present invention to facilitate generation of random-numbers in a bit-slice arrangement. “CA 150” is one classification of cellular automata using notation developed by Stephen Wolfram. In particular, CA150 corresponds to an XOR function with a neighborhood size of three and having a decimal value of 150 in the resulting-eight-row truth table (i.e., “10010110” is the truth table resulting from a 3 input XOR function). Accordingly, while one implementation of the present invention uses CA150 other implementations could use different CA and have similar contemplated results. For more information on different CA and the CA nomenclature, see “Cellular Automata Based Random Number Generators Designed for FPGA Implementation”, written by the inventors of the present invention Barry Shackleford and Motoo Tanaka and published in The 10^{th} Workshop on Synthesis and System Integration of Mixed Technologies, presented Oct. 18-19, 2001.

Mode select is used to switch the bit-slice from operating in one of three different modes: hold, initialization, and run by selecting binary combinations 00, 01, and 1? respectively where “?” can be either 0 or 1. Hold is used to stop processing in the GA circuit and retain certain values in storage useful in debugging. Initialization uses implementations of the present invention to create random numbers using CA 404 and fill population memory with randomly generated chromosomes. Run mode causes GA circuit to evaluate and identify an optimal solution once the population memory is filled with the initial set of randomly generated chromosomes in accordance with the present invention. Accordingly, initialization mode is selected once for each run to initially load population memory with high quality random numbers in accordance with implementations of the present invention. These random numbers in population memory are used subsequently during run mode.

In initialization mode, results from CA 404 are processed by crossover logic 408 and mutation logic 410 as previously described to further improve the quality of random numbers generated. The resulting randomly generated numbers are routed to population memory until the memory is full and capable of being evaluated.

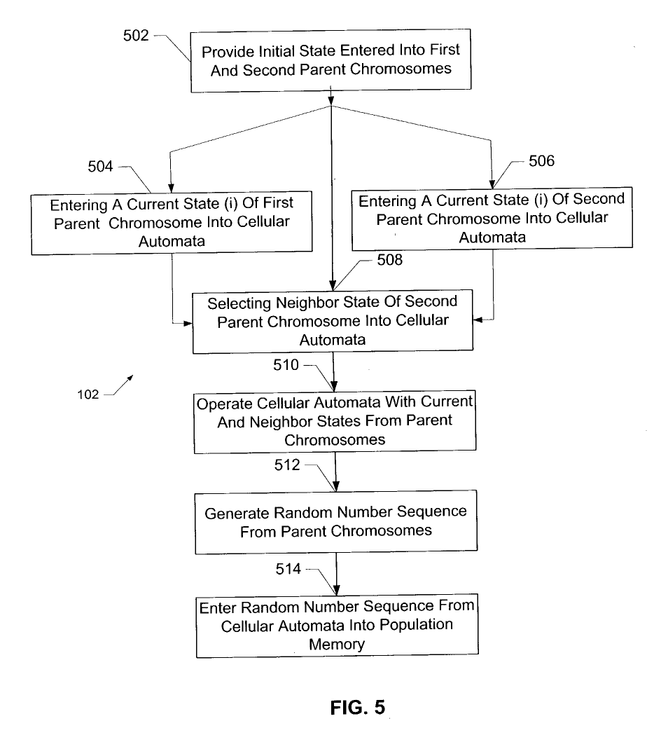

FIG. 5 is a flowchart diagram of the operations associated with generating the randomly generated numbers using cellular automata in accordance with one implementation of the present invention. Initially, an initial state is provided for both the first and second parent chromosomes (502). As previously described, in a bit-slice architecture this is accomplished using a randomly generated right or left shifting serial number stream. The initial state introduced in the first and second parent chromosomes can be any randomly generated value and operates as seed for further randomizations performed in accordance with implementations of the present invention. For example, the initial state can be one or more bits of information randomly generated through a simulated “coin-toss” type random number generator.

In one implementation, a current state of the first parent chromosome (504), a current state of the second parent chromosome (506) is entered into cellular automata. A neighbor state of the second parent chromosome is selected as a third input to the cellular automata (508) in this particular implementation. These inputs can be provided to a three-input XOR function otherwise known as CA150; neighbor state from the second parent chromosome can be provided synchronously with the other inputs or asynchronously depending on the particular CA150 implementation. Further, the neighbor state may also be taken from the first parent chromosome rather than the second parent chromosome and also depends on the design particulars.

Cellular automata operate on the current and neighbor states according to a particular logic function (510). In the case of CA 150, a three input XOR operates on eight input triplets producing the outputs as follows:

| XOR | |
|---|---|
| Inputs | XOR |
| P1, P2, N | Outputs |
| 000 | 0 |
| 001 | 1 |
| 010 | 1 |
| 011 | 0 |
| 100 | 1 |
| 101 | 0 |
| 110 | 0 |
| 111 | 1 |

Where:
P1: Current State (bit) of Parent 1
P2: Current State (bit) of Parent 2
N: Neighbor state

A sequence of these cellular automata linked together generate the desired random numbers from the initial parent chromosomes described previously (512). In one implementation, the results from the cellular automata are also processed by the crossover and mutation functions in the GA to further eliminate patterns within each chromosomes as well as correlations between different chromosomes. The resulting random number sequences produced by the cellular automata are eventually entered into the population memory (514) in preparation for GA processing.

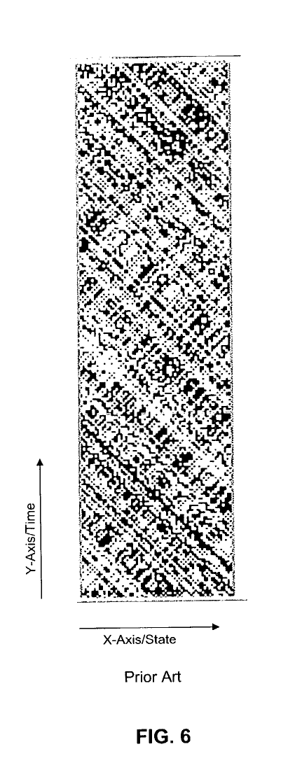

To validate the results, FIG. 6 provides a graphical representation of the random numbers generated without the use of cellular automata. State information of a bit-sequence is represented along the x-axis while the y-axis shows how the state information varies over time. Clear patterns in FIG. 6 indicate the number sequence has a certain degree of predictability and therefore does not exhibit high quality random behavior. This can be seen in both the state information along the x-axis as well as over time in the y-axis.

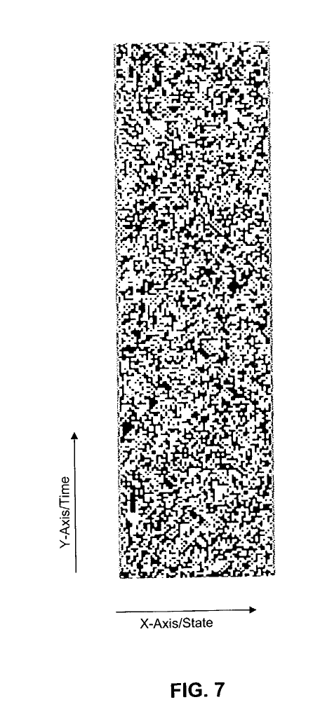

FIG. 7 graphically illustrates the results when using computer automata to develop random number sequences in accordance with one implementation of the present invention. In contrast to FIG. 6, FIG. 7 illustrates fewer patterns both in the state information along the x-axis and over time in the y-axis. Consequently, adding the computer automata logic assists in making number sequences more random and improves the chances of identifying near optimal and optimal solutions using GA analysis.

While examples and implementations have been described, they should not serve to limit any aspect of the present invention. Accordingly, implementations of the invention can be implemented in digital electronic circuitry, or in computer hardware, firmware, software, or in combinations of them. Apparatus of the invention can be implemented in a computer program product tangibly embodied in a machine-readable storage device for execution by a programmable processor; and method steps of the invention can be performed by a programmable processor executing a program of instructions to perform functions of the invention by operating on input data and generating output. The invention can be implemented advantageously in one or more computer programs that are executable on a programmable system including at least one programmable processor coupled to receive data and instructions from, and to transmit data and instructions to, a data storage system, at least one input device, and at least one output device. Each computer program can be implemented in a high-level procedural or object-oriented programming language, or in assembly or machine language if desired; and in any case, the language can be a compiled or interpreted language. Suitable processors include, by way of example, both general and special purpose microprocessors. Generally, a processor will receive instructions and data from a read-only memory and/or a random access memory. Generally, a computer will include one or more mass storage devices for storing data files; such devices include magnetic disks, such as internal hard disks and removable disks; magneto-optical disks; and optical disks. Storage devices suitable for tangibly embodying computer program instructions and data include all forms of non- volatile memory, including by way of example semiconductor memory devices, such as EPROM, EEPROM, and flash memory devices; magnetic disks such as internal hard disks and removable disks; magneto-optical disks; and CD-ROM disks. Any of the foregoing can be supplemented by, or incorporated in, ASICs.

While specific embodiments have been described herein for purposes of illustration, various modifications may be made without departing from the spirit and scope of the invention. Accordingly, the invention is not limited to the above-described implementations, but instead is defined by the appended claims in light of their full scope of equivalents.

What is claimed is:

1. A method of initializing a population memory of electronic chromosomes as used in genetic algorithm analysis, comprising:
providing an initial state into a first parent chromosome and a second parent chromosome;
entering a current state of a first parent chromosome and a second parent chromosome into a cellular automata;
selecting a neighbor state of the second parent chromosome for entry into the cellular automata; and
generating a random number sequence using the cellular automata for use in the population memory.

2. The method of claim 1 further comprising:
entering the entire population memory with the random number sequence generated using the cellular automata.

3. The method of claim 1 wherein the initial state in the first parent chromosome and the second parent chromosome is created using a shifting serial random number stream.

4. The method of claim 1 wherein the current state of the first parent chromosome and the current state of the second parent chromosome corresponds to the same bit-offset in both the first parent chromosome and second parent chromosome.

5. The method of claim 1 wherein the neighbor state of the second parent chromosome corresponds to at least a one-bit offset from the current state of the second parent chromosome.

6. The method of claim 1 wherein the cellular automata corresponds to a three-input exclusive-or (XOR) operation.

7. A computer program product for initializing a population memory of electronic chromosomes as used in genetic algorithm analysis, tangibly stored on a computer-readable medium, comprising instructions operable to cause a programmable processor to:
provide an initial state into a first parent chromosome and an initial state in a second parent chromosome;
enter a current state of a first parent chromosome and a current state of a second parent chromosome into a cellular automata;
select a neighbor state of the second parent chromosome for entry into the cellular automata; and
generate a random number sequence using the cellular automata for use in the population memory.

8. The computer program product of claim 7 further comprising instructions to:
enter the entire population memory with the random number sequence generated using the cellular automata.

9. The computer program product of claim 7 wherein the initial state in the first parent chromosome and the second parent chromosome is created using a shifting serial random number stream.

10. The computer program product of claim 7 wherein the current state of the first parent chromosome and the current state of the second parent chromosome corresponds to a bit-offset in both the first parent chromosome and second parent chromosome.

11. The computer program product of claim 7 wherein the neighbor state of the second parent chromosome corresponds to at least a one-bit offset from the current state of the second parent chromosome.

12. The computer program product of claim 7 wherein the cellular automata corresponds to a three-input exclusive-or (XOR) operation.

13. An apparatus for initializing a population memory of electronic chromosomes as used in genetic algorithm analysis, comprising:
means for providing an initial state into a first parent chromosome and an initial state in a second parent chromosome;
means for entering a current state of a first parent chromosome and a second parent chromosome into a cellular automata;
means for selecting a neighbor state of the second parent chromosome for entry into the cellular automata; and
means for generating a random number sequence using the cellular automata for use in the population memory.

14. An apparatus for initializing a population memory of electronic chromosomes as used in genetic algorithm analysis, comprising:
a first parent circuit for receiving a parent chromosome from a population memory;
a second parent circuit for receiving a parent chromosome from a population memory; and
a cellular automata circuit that receives a first current state from a first electronic chromosome, a second current state from a second electronic chromosome, and a neighboring state from the second electronic chromosome.

15. The apparatus of claim 14 wherein the first parent circuit and the second parent circuit for receiving the parent chromosomes are each initially loaded with a serial random number stream using a shifting register.

16. The apparatus of claim 14 wherein the cellular automata is implemented using a three-input exclusive-or (XOR) circuit.

17. The apparatus of claim 14 wherein the neighboring state from the second electronic chromosome is at a bit-offset from the first current state and the second current state.

18. The apparatus of claim 17 wherein the bit-offset in the first electronic chromosome used to select the first current state corresponds to the offset in the second electronic chromosome used to select the second current state.

19. The apparatus of claim 17 wherein the bit-offset used to select the neighboring state from the second electronic chromosome is at least a 1-bit difference from the first current state and the second current state.

20. The apparatus of claim 14 implemented as a bit-slice that can be combined and used in an n-bit wide genetic algorithm analysis circuit.

21. The apparatus of claim 14 implemented within an n-bit wide genetic algorithm circuit.

22. The apparatus of claim 14 further comprising a mode select circuit that directs the random number sequence from the cellular automata into the population memory during an initialization sequence of a genetic algorithm circuit.
